(19) United States
(12) Patent Application Publication
(10) Pub. No.: US 2021/0085298 A1
(43) Pub. Date: Mar. 25, 2021

(54) CORE BIOPSY INSTRUMENT

(71) Applicant: URO-1, Inc., Winston-Salem, NC (US)

(72) Inventors: Kevin Joseph Rackers, Greensboro, NC (US); Philip Allred, III, Kernersville, NC (US); Phillip Jack Snoke, Winston-Salem, NC (US); Bela Denes, Jacksonville, OR (US); Ted Belleza, La Selva Beach, CA (US)

(73) Assignee: URO-1, Inc., Winston-Salem, NC (US)

(21) Appl. No.: 16/725,840

(22) Filed: Dec. 23, 2019

Related U.S. Application Data
(60) Provisional application No. 62/973,168, filed on Sep. 23, 2019.

(51) Int. Cl. A61B 10/02

(52) U.S. Cl. CPC A61B 10/0241

(57) ABSTRACT

A core biopsy instrument for taking samples of tissue such as prostate tissue comprises a handle configured to releasably receive a cartridge that holds a core collector. In use, after loading a cartridge in the handle, a spring drives the core collector into target tissue and shortly thereafter, in less than a second, a spring drives a cannula over the core collector to cut tissue in a trough thereof from surrounding tissue. The core collector is withdrawn proximally to its initial position in the cartridge and the cartridge is removed from the handle. The cartridge, with the core collector and the tissue sample therein is treated as needed for sending as a unit to a laboratory, thus preserving the orientation and completeness of the tissue sample therein.

### REFERENCE TO RELATED APPLICATION

This application claims priority to provisional patent application Ser. No. 62/973,168 filed Sep. 23, 2019 and incorporates by reference the contents of said provisional application.

### FIELD

This patent specification relates to medical devices for taking tissue samples. More particularly, some embodiments relate to an integrated, handheld, low-cost core biopsy instrument that is particularly suitable for taking samples of prostate tissue.

### BACKGROUND

When suspicious tissue is discovered in a patient's prostate or in another area through manual examination or an imaging modality such as ultrasound, MRI, X-ray imaging or the like, it may be desirable to perform a biopsy procedure to remove one or more samples of that tissue to help determine if the tissue contains cancerous cells, other cells of interest such as precancerous other cells, or to gain other information. A biopsy may be performed using an open or a percutaneous method. For the prostate tissue, typically a core needle device is used that enters the prostate transrectally (TRUS) or transperineally (TPUS). There are complex prostate sampling devices that require two or more people for the procedure, and there are simpler devices that can even be disposed after use on one patient. U.S. Pat. Nos. 5,546,957, 5,526,822, and published U.S. Patent application US 2016/0166331 A1 discuss examples of biopsy devices. The contents of said patents and published application are hereby incorporated by reference in this patent specification. A core biopsy device called Bard Max-Core is believed to be commercially available from BD Bard in Tempe, Ariz.

This patent specification is directed to biopsy instruments that overcome several shortcomings of the known biopsy instruments, as discussed in detail below.

### SUMMARY OF THE DISCLOSURE

A new core biopsy instrument according to some embodiments comprises a handle; a cannula having a proximal portion secured to the handle and a distal end and configured to move distally and proximally relative to the handle along a central axis; a drive rod having a distal portion configured to move distally and proximally along said central axis along a path coaxial with said cannula; a core collector having a distal end, a proximal portion, and a trough intermediate said distal end and said proximal portion, wherein said trough comprises two sidewalls terminating in respective rows of teeth alternating along said central axis; a cartridge releasably holding said core collector; said cartridge and handle being configured for releasable attachment to each other in which said core collector extends along said central axis and is coaxial with said cannula; said drive rod having a distal portion configured to interlock with said proximal portion of the core collector as said cartridge is being releasably attached to said handle; said drive rod being spring loaded and configured when interlocked with said core collector to drive the core collector distally inside said cannula to a tissue sampling position in which the core collector distal end extends distally from the cannula over a selected distance; said cannula being spring loaded and configured to respond to the drive rod having driven the core collector to said tissue sampling position to move distally over the core collector to a position at which the distal ends of the cannula and of the core collector are aligned; said distal ends of the core collector and the cannula conforming to respective planes inclined at opposite angles to said central axis; and said drive rod being further configured to pull said core collector proximally from said sampling position to a position in which the core collector is held in said cartridge and said cartridge being configured to be released from the handle while carrying said core collector.

According to some embodiments: the teeth can have have proximal sides that are inclined proximally relative to said central axis or are normal to the central axis; the core collector has holes or indentations in a proximal portion thereof and the cartridge includes one or more resilient members that hold the core collector in place in the cartridge but bend back to allow the core collector to move distally and proximally along said central axis when pushed or pulled by said drive rod interlocked therewith; the teeth at one side of said trough are at cross sections of the core collector at which there are no teeth on the other side of the trough so that in a cross section normal to the central axis and intersecting one of said teeth one of the sides of the trough is taller than the other; and respective springs housed in said handle are configured to selectively drive distally said core collector and thereafter said cannula.

According to some embodiments, a core biopsy instrument comprises: a tubular cannula extending along a central axis and having a sharp distal end; a core collector coaxial with said cannula and configured to slide inside the cannula distally and proximally, said core collector having a distal portion, an intermediate portion and a proximal portion, wherein: the distal portion of the core collector terminates distally in a sharp end; the intermediate portion of the core collector comprises a trough having side walls terminating in teeth spaced apart along said central axis; and the proximal portion of the core collector has interlock portions of differing cross sectional areas at different positions along said central axis; and the core biopsy device further comprises a drive rod confined to move distally and proximally along said central axis and having a distal portion with cross sections of different areas that are shaped and dimensioned to interlock with said interlock portions of the core collector. The distal end of said core collector can have a distal face conforming to a plane inclined in one direction relative to the central axis while the distal end of the cannula can conform to plane included in an opposite direction to the central axis; the instrument can further include. a handle to which said core collector and said cannula are secured for proximal and distal motion along said central axis relative to the handle and to each other; and. a cartridge releasably secured to said handle, wherein said core collector has indentations or holes in said proximal portion thereof and said cartridge is configured to hold said core collector when aligned therewith so that the cartridge is removed from the handle together with said core collector.

According to some embodiments, a method comprises introducing a tubular cannula and a core collector into tissue while distal ends thereof are aligned along a central axis; driving a selected length of the core collector distally from the cannula and into tissue to thereby collect an elongated sample of tissue in a trough in said core collector formed by walls terminating in spaced apart and staggered rows of teeth extending along said central axis; thereafter, driving the cannula over said core collector to thereby sever from surrounding tissue said sample of tissue in said trough of the core collector; pulling the core collector with said tissue sample therein proximally to a position aligned with a cartridge that is releasably secured to the handle and releasably latching the core collector to the cartridge; and removing the cartridge with the core collector latched thereto from said handle.

### BRIEF DESCRIPTION OF THE DRAWINGS

FIG. 1 is a partly schematic side view of a core biopsy instrument according to some embodiments.

FIG. 2a is a side view of a core collector and FIGS. 2b-2d are perspective views according to some embodiments.

FIG. 3 is otherwise like FIG. 1 but shows a cartridge releasably secured in a handle according to some embodiments.

FIG. 4 is otherwise like FIG. 3 but shows a core collector advanced into a cannula according to some embodiments.

FIG. 5 is otherwise like FIG. 4 but shows a core collector advanced distally out of a cannula according to some embodiments.

FIG. 6 is otherwise like FIG. 5 but shows a cannula advanced distally over a core collector according to some embodiments.

FIG. 7 is an enlarged side view of a distal portion of a core collector and a sectional view of a cannula partly advanced over the core collector according to some embodiments.

FIG. 8 is a perspective view of the portions of a cannula and core collector seen in FIG. 7 according to some embodiments.

FIG. 9 is a perspective view of a cartridge according to some embodiments.

FIG. 10 is an enlarged perspective view of a cartridge according to some embodiments.

FIG. 11 is a side view of a drive rod interlocked with a proximal portion of a core collector according to some embodiments.

FIG. 12 is a perspective view of a drive rod interlocking with a proximal portion of a core collector according to some embodiments.

FIG. 13a is a side view, FIG. 13b is a plan view, and FIGS. 13c and 13d are perspective views of a distal portion of a drive rod according to some embodiments.

FIGS. 14a and 14b are sectional views along planes D and E respectively of FIG. 4, and FIGS. 14c-14e are sectional views along planes F-H respectively of FIG. 7.

FIG. 15 is a perspective view of a core biopsy instrument according to some embodiments.

FIGS. 16a-16d schematically illustrate steps in the operation of a spring loaded cannula and a spring loaded core collector according to some embodiments

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description in order to provide a thorough understanding, some embodiments can be practiced without some or all these details. Moreover, for the purpose of clarity, certain technical material that is known in the related art has not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

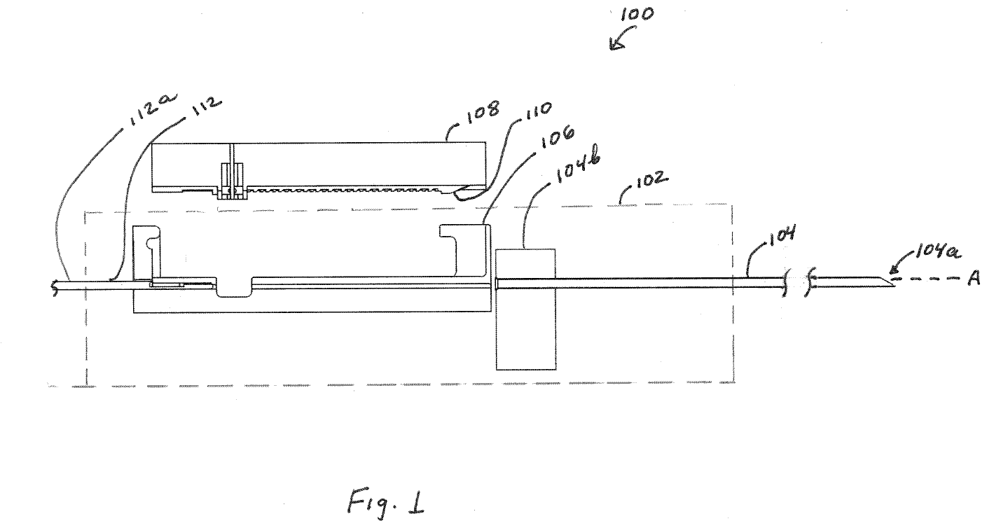

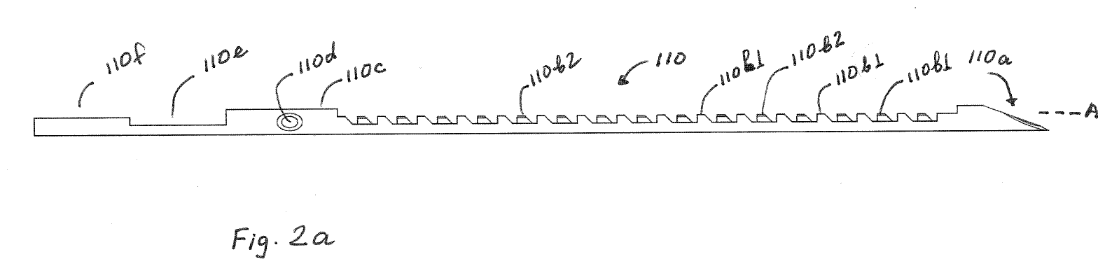

FIG. 1 illustrates a core biopsy instrument 100 according to some embodiments. The instrument comprises a housing or handle 102 that is only schematically illustrated, from which a tubular cannula 104 extends distally and terminates in a sharp distal end 104a formed by cutting cannula 104 along a plane angled relative to a longitudinal central axis A. Cannula 104 has a proximal end held in a support block 104b that rides in a channel or along a guide rod or rods (not shown) inside handle 102 for motion along axis A both distally and proximally. A holder block 106 that is in a fixed position in handle 102 and is configured to releasable accept a cartridge 110 that holds a core collector 110. A drive rod 112 rides in a channel (not shown) in holder 106 that extends along central axis A, and a proximal portion 112a of drive rod 112 may extend proximally from a proximal end of handle 102 and may terminate proximally in a handle or knob (not shown) for manually moving rod 112 distally and proximally relative to handle 102. Drive rod 112 is constrained in handle 102 to move distally and proximally along central axis A.

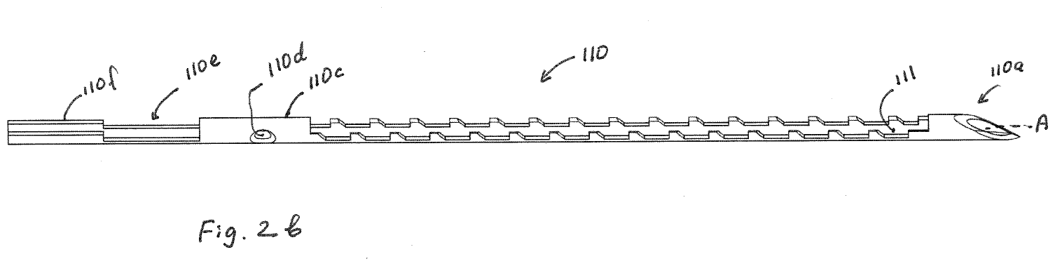

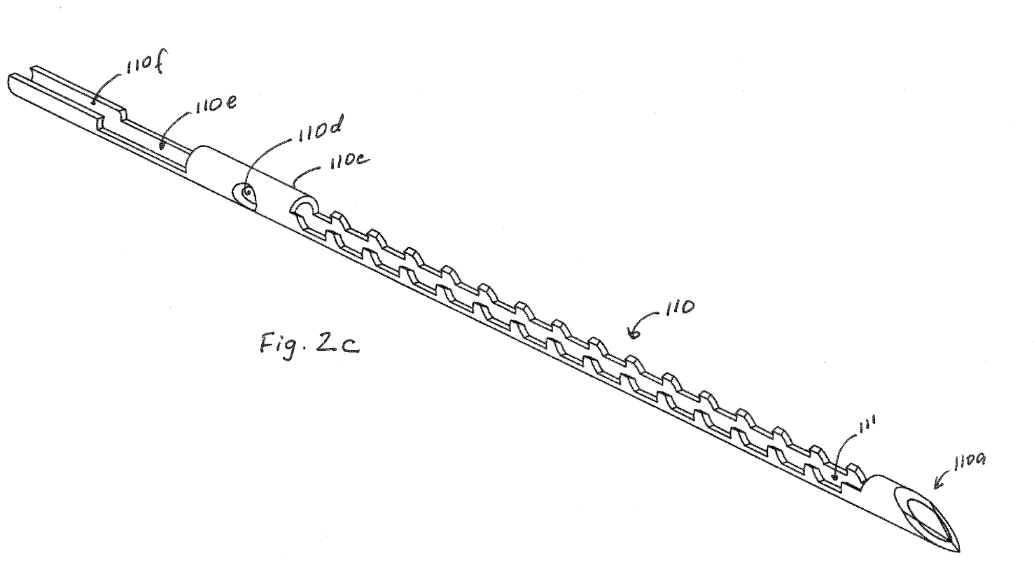

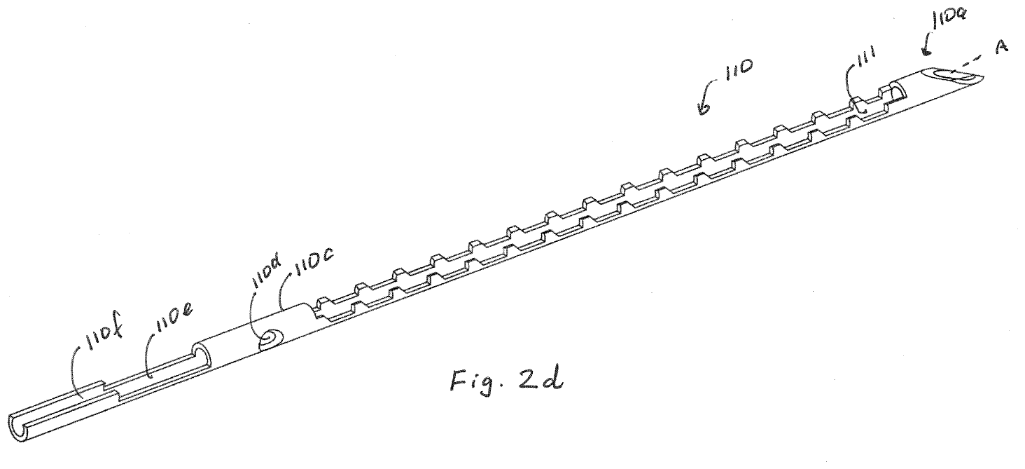

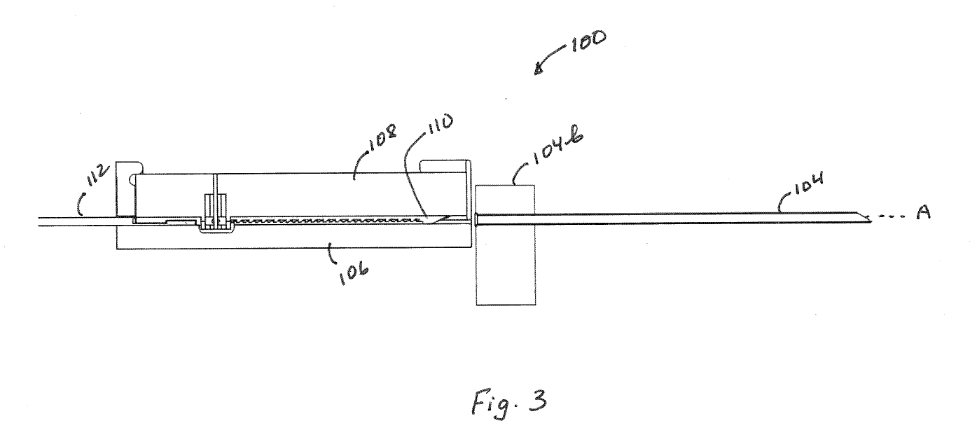

FIG. 2a is a side view of core collector 110 and FIGS. 2b-3d are different perspective views of the core collector according to some embodiments. Core collector 110 has a sharp distal end 110a that conforms to a plane angled to axis A and preferably is closed to no tissue would enter along axis A through that distal end. Two rows of teeth 110b1 and 110b2 extend proximally from distal end 110a of core collector 110. In cross-section normal to axis A, the portion of core collector 110 with the rows of teeth is roughly semicircular to define a trough 111 (FIGS. 2b-2d) with sidewalls from which teeth 110a and 110b extend. The teeth have proximal sides that can be inclined in the proximal direction and sharpened to serve an important function described further below. A tubular portion 110c of core collector 110 is proximal to the two rows of teeth and has side holes 110d that serve a function explained further below. A cutout 110e is proximal to tubular portion 110d and in cross-section normal to axis A is an arc that preferably extends over less than 180 degrees. The proximal end 110f of core collector 110 also is an arc but over a greater angle, preferably more than 180 degrees, so that cutout 110a ends in a step up proximally and distally formed by portions 110d and 110f.

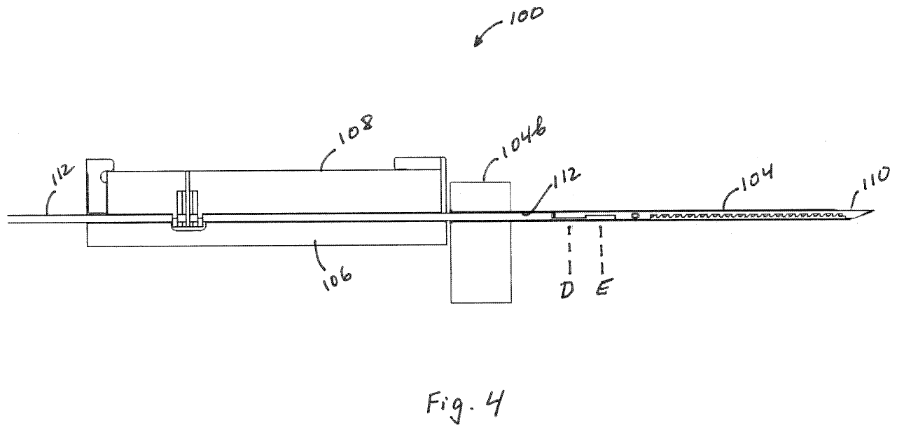

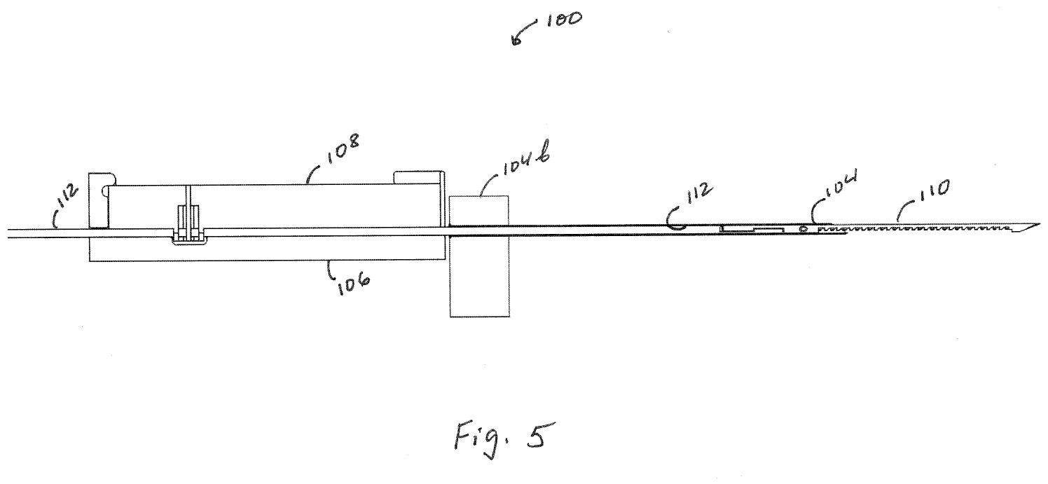

FIGS. 3-6 are partly side views and partly sectional views of core biopsy instrument 100 that illustrate its operation in some embodiments. For clarity, handle 102 is omitted from these views but it is present when using the instrument. FIG. 3 illustrates an initial state, in which cartridge 108 has been releasably snapped into holder 106, such that core collector 110 is in a channel in holder 106 and is held in place, as described in more detail below, interlocked with a distal end of drive rod 112. FIG. 4 illustrates a state in which drive rod 112 has pushed core collector 110 distally such that its sharp distal end protrudes distally from cannula 104. For this purpose, drive rod 112 can be pushed distally manually from behind the proximal portion of handle 102. As discussed in more detail further below, the planes to which the distal ends of cannula 104 and core collector 110 conform are angled relative to central axis A in opposite directions. The distal end 110a of core collector 110 fits snugly in cannula 104 to keep tissue from entering space between them. In this state, a physician inserts cannula 104 and core collector 110 in a patient's tissue until the distal tip 110a of core collector 110 is at a desired position relative to the tissue to be sampled—for example, just outside the patient's prostate or just inside the prostate, or up to or just into a suspected lesion. This desired position can be determined based on clinical experience or by feel or by using imaging devices such as an ultrasound probe that can but need not be attached to instrument 100 or by use of other imaging modalities. FIG. 5 illustrates a state in which core collector 110 has been advanced distally relative to cannula 104 such that all or at least some of the two rows of teeth are distal from the distal tip of cannula 104. For this purpose, drive rod 112 can be spring loaded as discussed further below such that releasing the spring action drives rod 112 distally, thus pushing core collector 110 out of cannula 104 over a desired distance that can be set by a suitable stop (not shown) for drive rod 112 in handle 102. In this state, core collector 110 has penetrated the tissue to be sampled (not shown) and a sample of tissue has entered trough 111. The angle of the plane to which the sharp distal end 110a of core collector 110 conforms helps drive tissue in trough 111 because driving the distal end 110a of core collector 110 into tissue compresses more tissue that is on the open side of trough 111 (down as viewed in FIG. 5) and this compressed tissue tends to spring back up into trough 111. FIG. 6 illustrates a state reached shortly (preferably less than a second) after core collector 110 has been advanced distally to the state of FIG. 5. To reach the state of FIG. 6, cannula 104 also preferably is spring-loaded such that its spring action is released shortly after the spring action for core collector 110 is released, to drive cannula 104 distally over core collector 110. This distal motion of cannula 104 cuts from surrounding tissue the sample of tissue that has entered trough 111 of core collector 110. In this cutting action, the two rows of teeth help keep the tissue sample from bunching up or exiting trough 111 as described in more detail further below. Thereafter, both cannula 104 and core collector 110, in their respective positions seen in FIG. 6, are pulled proximally, for example by manually pulling proximally drive rod 112 and holder block 106 holding cannula 104, until cannula 104 and core collector 110 are in the positions seen in FIG. 4, and drive rod 112 is pulled further proximally until it brings core collector 110 to the position seen in FIG. 3. Then, cartridge 108, which has gripped core collector 110 (with the tissue sample therein) as described further below, is manually pulled away from handle 102, and processed chemically and sealed, for example for sending to a laboratory. Notably, the tissue sample remains in cartridge 108 and remains undisturbed by handling. The orientation of the tissue sample relative to the living tissue from which the sample was extracted also is preserved due to core collector remaining in cartridge 106, as the distal end of cartridge 106 is known, so the laboratory would have an unambiguous indication or which end of the tissue sample is distal and which is proximal.

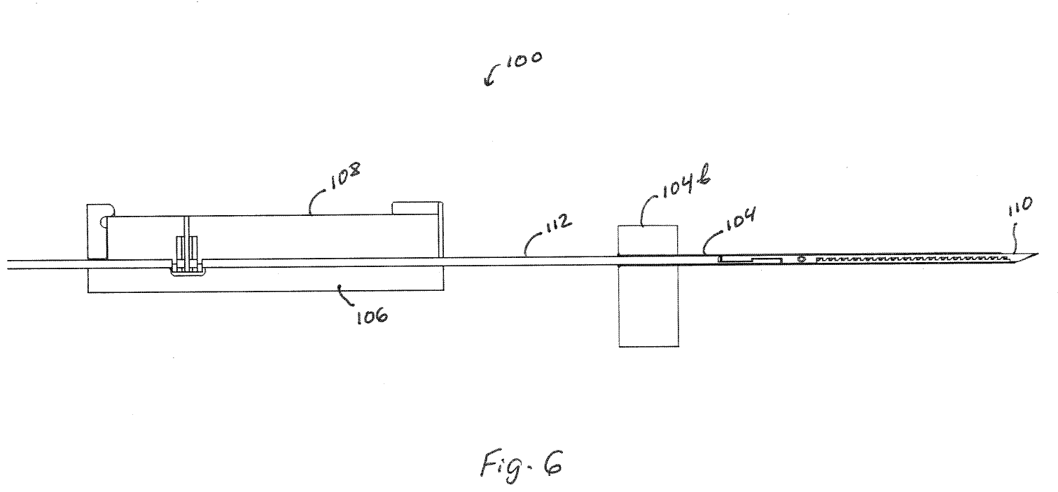

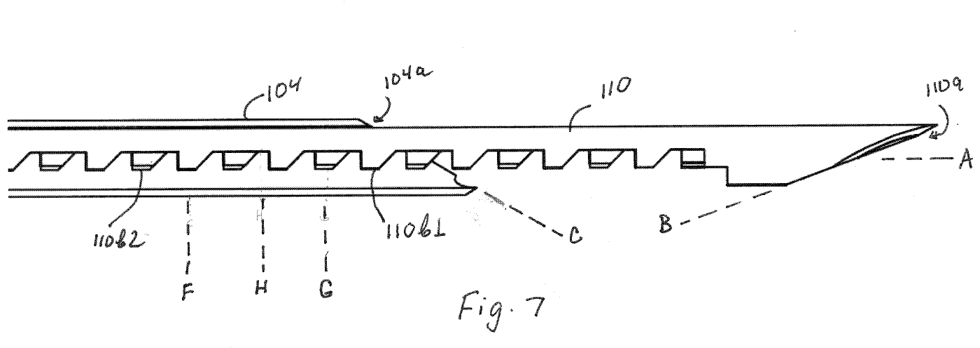

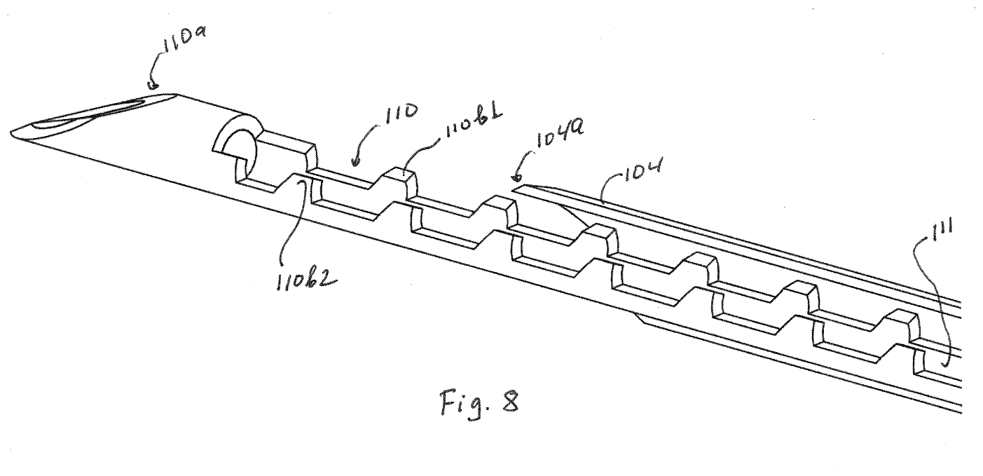

FIG. 7 is a partial side view of a distal portion of core collector 110 and a sectional view of a distal position of cannula 104 and FIG. 8 is a perspective view of the same portions of cannula 104 and core collector 110. As seen in FIG. 7, the distal end 110a of core collector 110 conforms to a plane B that is inclined in one direction to central axis A and the distal end 104a of cannula 104 conforms to a plane C inclined in an opposite direction to central axis A. As seen in FIGS. 7 and 8, distal end 104a of cannula 104 is sharpened by gradually reducing the thickness of its wall in the distal direction until it reaches the inside diameter of cannula 104.

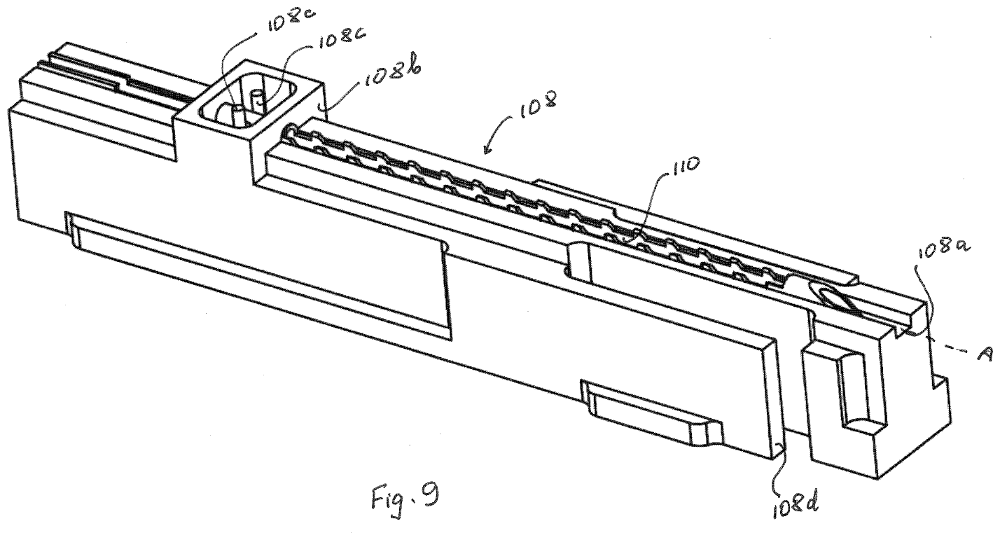

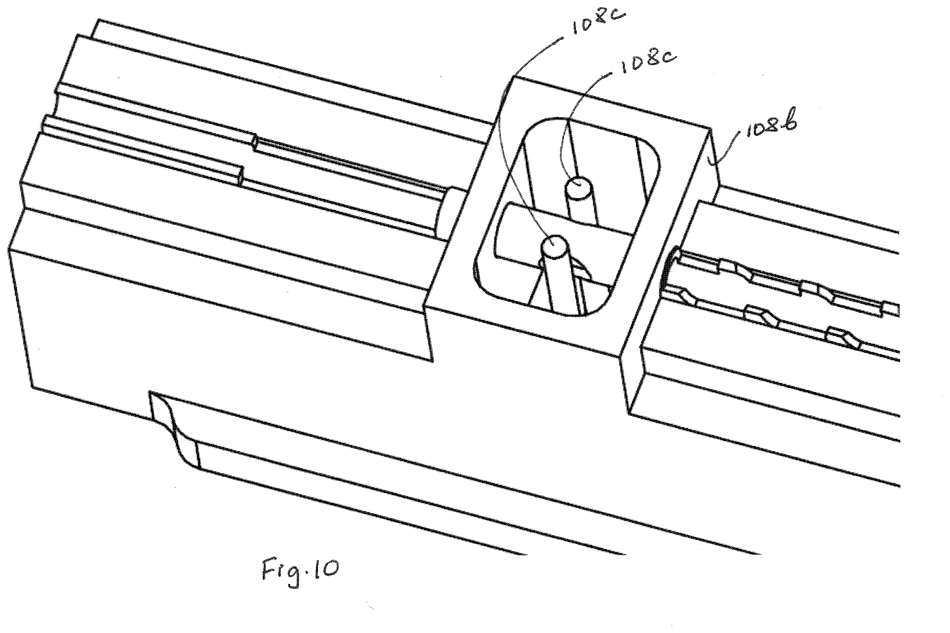

FIG. 9 is a perspective view of cartridge 108 and FIG. 10 is a perspective view of a portion of the cartridge as seen from a different viewpoint according to some embodiments. Cartridge 108 comprises a channel 108a that extends along central axis A and is dimensioned to snugly receive core collector 110 but allow it to move distally and proximally in the channel. A proximal portion of cartridge 108 has a cage 108b with openings through which core collector 110 passes and two pins 108c that are resilient and pinch into holes 110d (FIGS. 2a-2d) of core collector 110 when the core collector is in cartridge 108 in the position seen in FIGS. 9 and 10. Pins 108c are sufficiently resilient to allow drive rod 112 to push core collector 110 distally out of engagement with pins 108c and also to allow drive rod 112 to pull core collector 110 proximally to the position seen in FIGS. 9 and 10, at which pins 108a can again grip the core collector and keep it in place while cartridge 108 is pulled out of handle 102. Cartridge 108 has provisions such as a cantilevered projection 108d to releasably snap into secure engagement with corresponding projections or indentations (not shown) in handle 102.

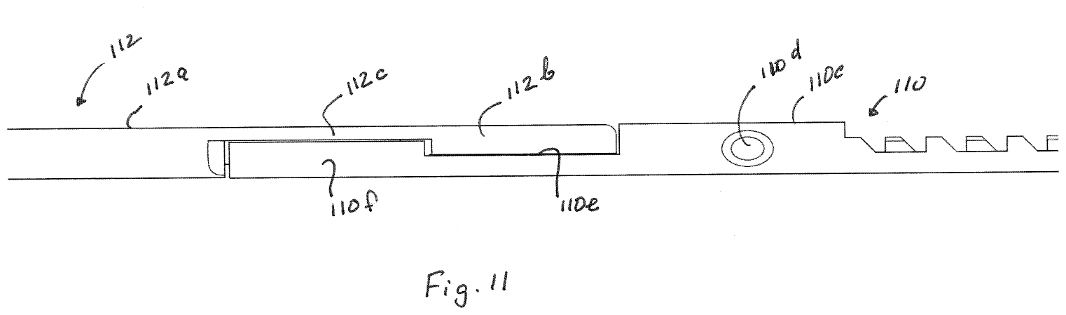

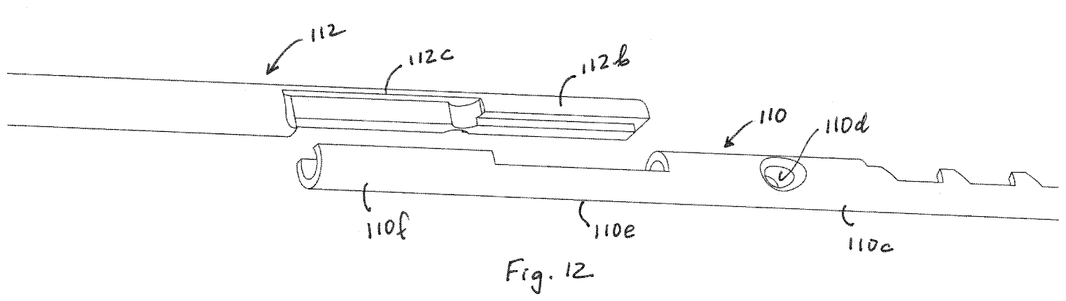

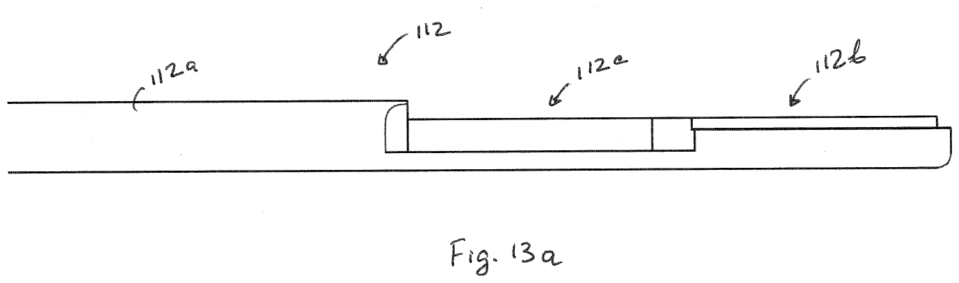

FIG. 11 is a side view illustrating an interlock of drive rod 112 and core collector 110 and FIG. 12 is a perspective view of the proximal end of core collector 110 and the distal end of drive rod 112 according to some embodiments. Drive rod 122 has a thicker distal end 112b that fits in the smaller-arc cutout 110e of core collector 110 and a thinner portion immediately proximal that fits into the larger-arc cutout 110f in core collector 110. In operation, the distal end of drive rod 112 is in its position seen in FIG. 1 when cartridge 108 is not yet inserted in handle 102. Inserting cartridge 108 to the position seen in FIG. 3 places drive rod 112 and core collector 110 in the interlocked position seed in FIG. 11. Removing cartridge 108 by pulling it from handle 102 disengages core collector 110 from interlock with drive rod 110 while drive rod 112 remains in place in handle 102.

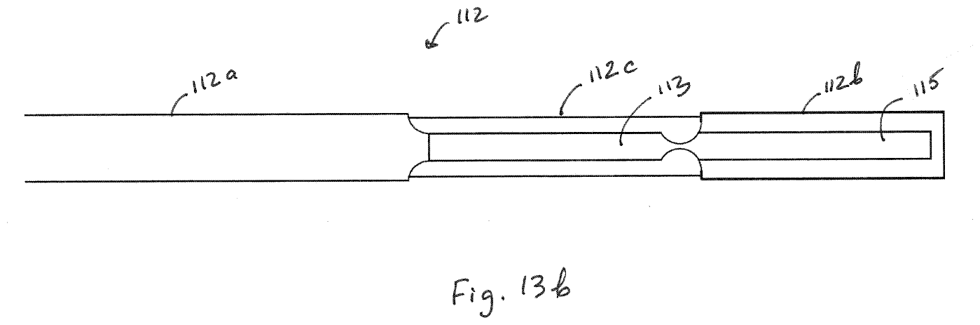

FIG. 13a is a side view of a distal portion of drive rod 112, FIG. 13b is a plan view, and FIGS. 13c and 13d are two different perspective views. Portion 12c of drice rod 112 comprises a thinner band on one side of a stiffening rib 113, and portion 112b comprises a wider arc and a thinner rib 115, all serving to provide an interlock with core collector 110.

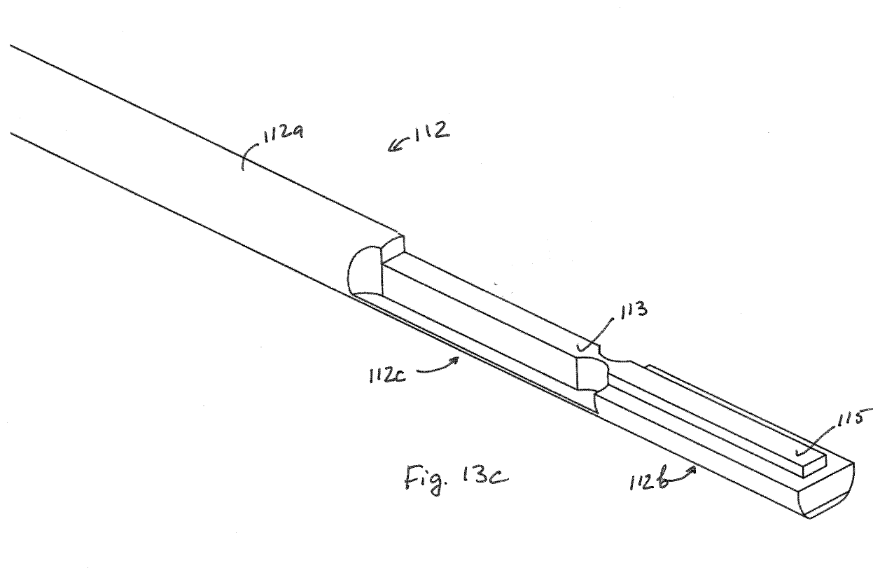

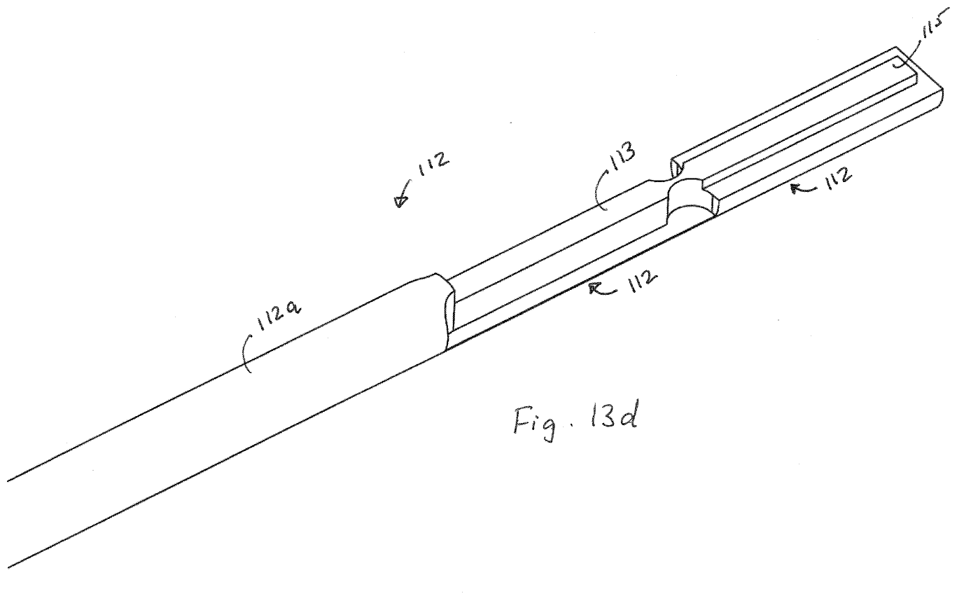

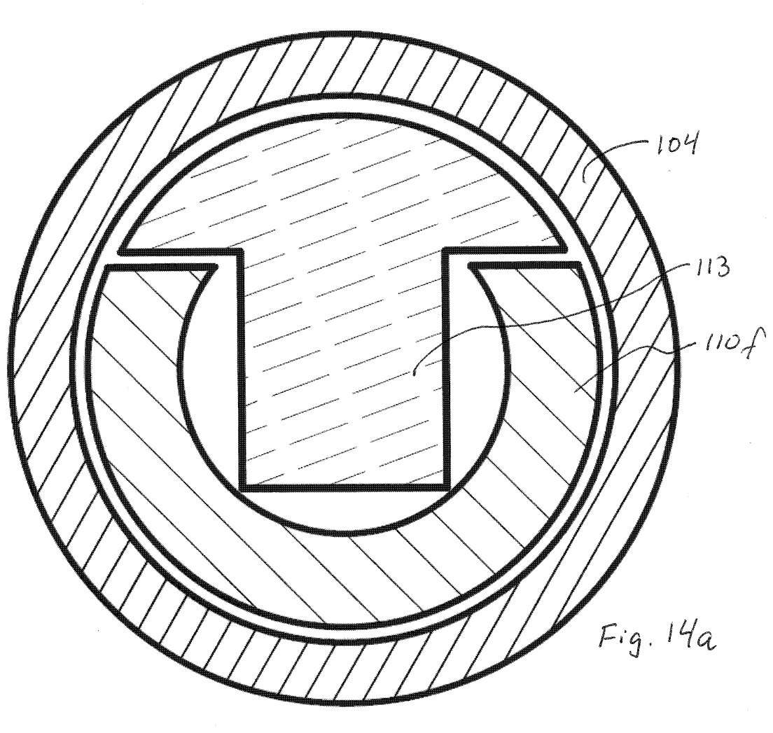

FIGS. 14a-14e illustrate cross-section of core collector 110 inside cannula 104 at respective planes normal to central axis A. FIG. 14a is a section along plane D in FIG. 4 and illustrates how stiffening rib 113 (FIGS. 13b-13d) fits in portion 110f (FIG. 12) of core collector 110 when surrounded by cannula 104 in the state shown in FIG. 12. FIG. 14b is a section along plane E of FIG. 4 an illustrates how rib 115 (FIGS. 13b-13d) fits in portion 110f (FIG. 12) of core collector 110 that is in cannula 104 in the state shown in FIG. 12. FIG. 14c illustrates a section along plane F in FIG. 7, and shows a tooth 110b2 at right (as viewed in FIG. 14c) that extends above the left sidewall of trough 111 of core collector 110 in that section. FIG. 4d illustrates a section along plane G in FIG. 7 showing a tooth 110b1 at left (as viewed in FIG. 14d) that extends above the right sidewall of core collector 110 in that section. FIG. 14e illustrates a section along plane. H in FIG. 7, where there are no teeth and shows both sidewalls of trough 111 at same height.

Notably, as seen for example in FIGS. 7 and 8, the two rows of teeth are staggered relative to each other—where in a cross-section normal to central axis A there is a tooth 110b1 but no tooth 110b2 and where there is a tooth 110b2 there is no tooth 110b1. This can facilitate tissue enter, and stay in, trough 111 compared a trough extending over and arc like that of arcs of FIGS. 14c and 14d but lacking teeth.

In addition, as seen for example in FIG. 8, the distal sides of the teeth can be inclined in the proximal direction, and in some embodiments the proximal sides of the teeth also can be be somewhat inclined in the proximal direction. In other embodiments, the proximal sides of the teeth need not be inclined in the proximal direction or can be inclined in a different direction.

Notably, the teeth can be cut such that they have sharp edges on their proximal sides. Having the distal sides of the teeth inclined reduces the portion of a sidewall of trough 111 that is occupied by teeth and thus facilitate entry of tissue in the trough. Having the proximal sides of the teeth inclined in the proximal direction and/or formed with sharp edges facilitates severing of the tissue sample in trough 111 as cannula 104 moves distally over core collector 110.

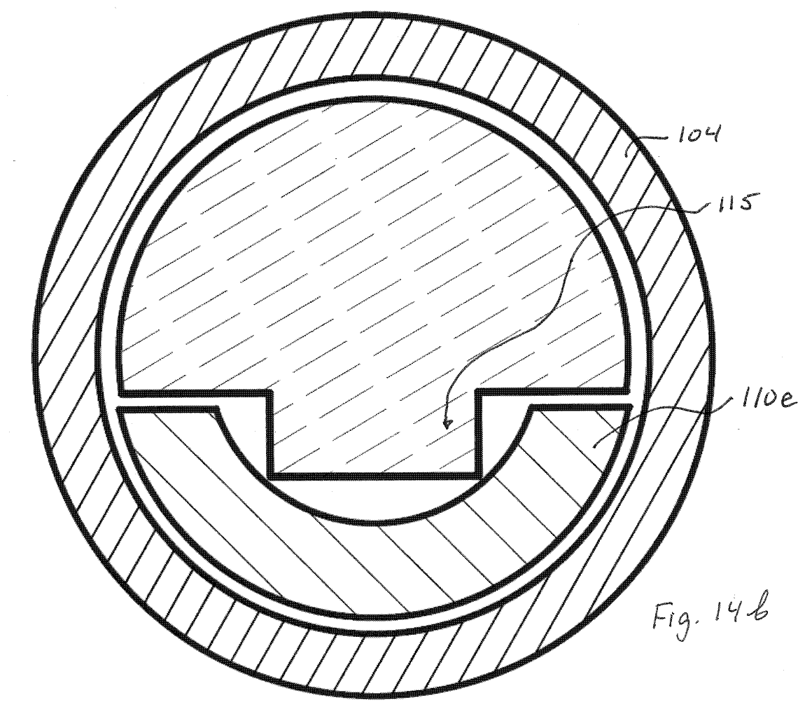

FIG. 15 is a perspective view of a core biopsy instrument 100 that shows an example of a handle 102, cannula 104 and a distal portion of core collector 110 protruding distally from cannula 104. FIG. 5 illustrates a knob 101 at the proximate side of handle that is affixed to the proximal end of drive rod 112 to manually pull the rod proximally and push it distally as needed. In addition. Knob 101 can be connected to springs (not shown) in handle 102 to compress them when knob 101 is pulled proximally until the springs latch in a compressed state. These springs when released drive distally core collector 110 and cannula 104 as described above. FIG. 15 also shows a manually operated switch 103 that when pressed releases from a latched state the compressed spring that drives rod 112 distally. The spring the drives cannula 104 distally can be released by a delay actuated by forward motion of rod 112 when driven by the spring releases by actuating switch 103. In a specific and non-limiting example, the approximate dimensions of handle 102 are: length 9.0 inches, width 2.2 inches, and height 1.3 inches. In this example, cannula 104 protrudes distally from handle 102 approximately 6 inches.

In one mode of operation, cannula 104 can be withdrawn from the patient after instrument 100 has reached the state illustrated in FIG. 6 or has reverted to the state of FIG. 4 after having reached the state of FIG. 6. For another tissue sample, the same instrument or an unused duplicate can be used as described above to take another tissue sample. Alternatively, after reaching the state of FIG. 6 or after reverting to the state of FIG. 4, cannula 104 can be left in the patient or moved some to a new orientation and/or depth in the patient, and only core collector 110 can be withdrawn into cartridge 108, the cartridge can be removed from handle 102 and replaced with a new cartridge, with a new core collector, and the procedure discussed above can be repeated to take a new tissue sample. Cartridges can be removed and replaced plural times while cannula 104 remains in the patient to thereby take multiple samples of tissue.

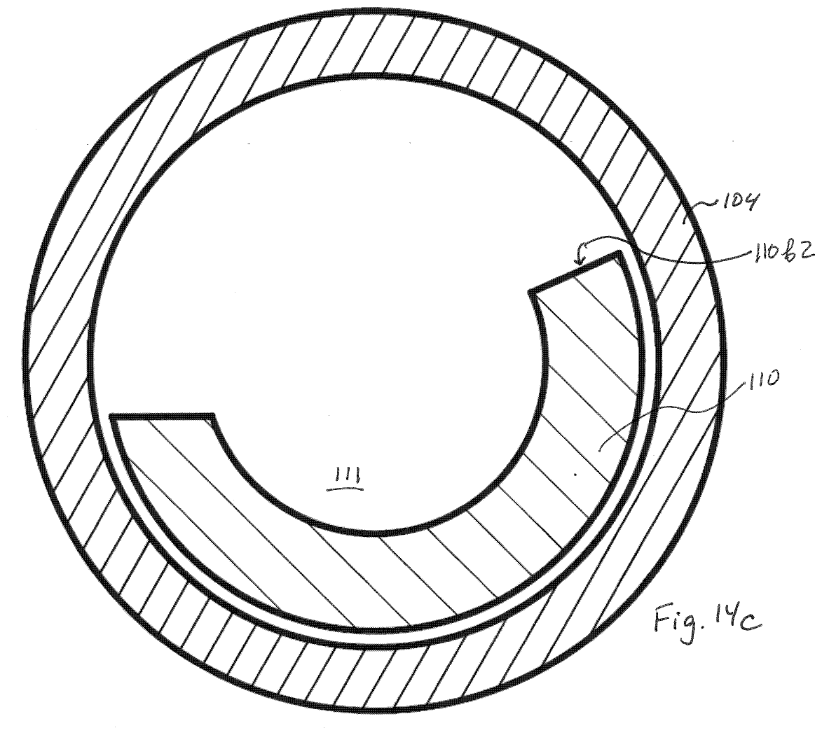

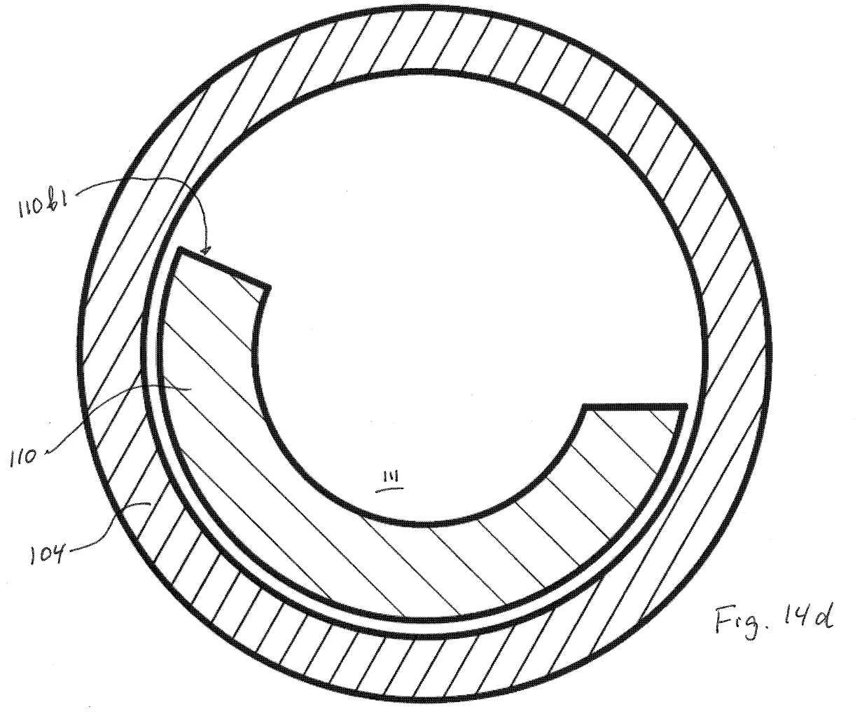

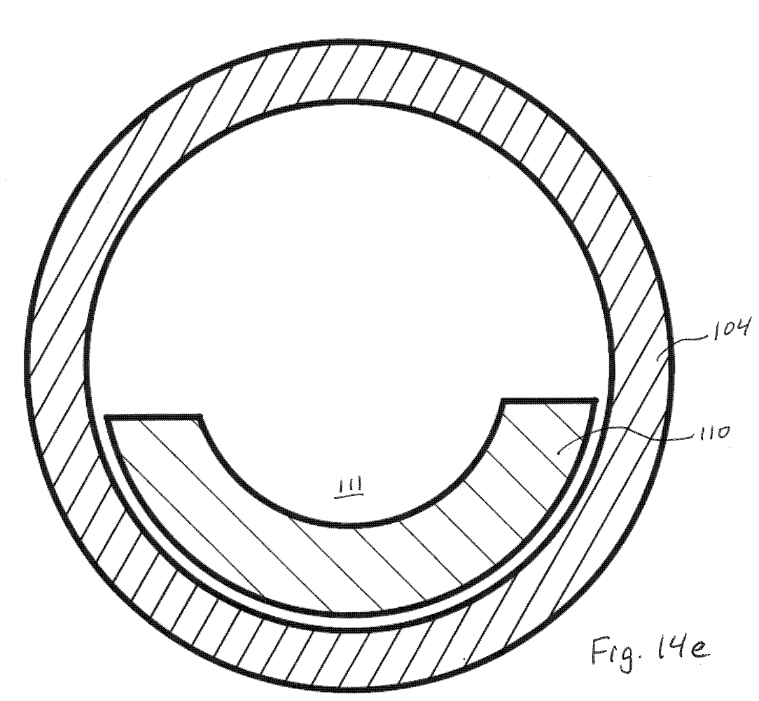

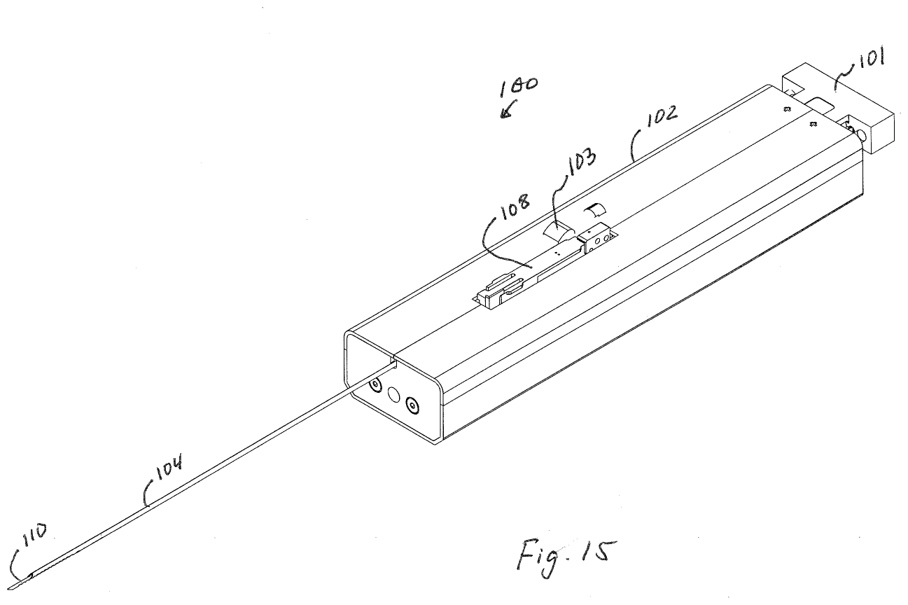

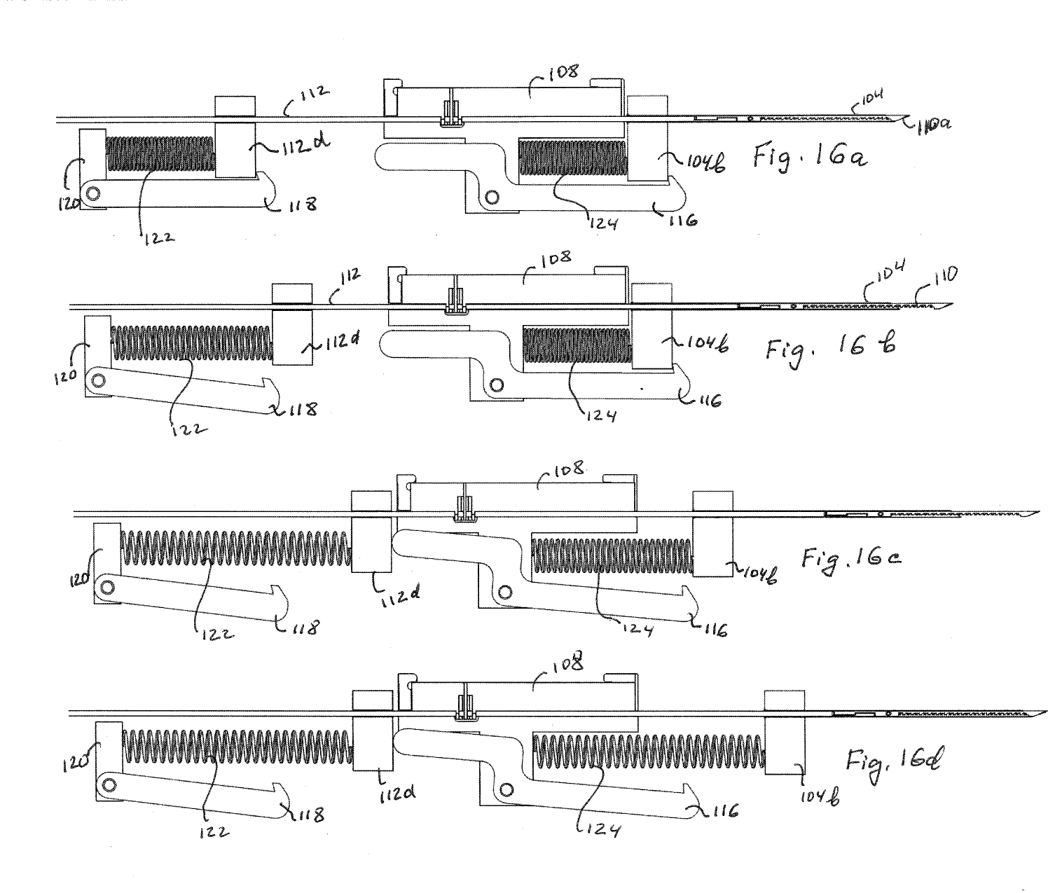

FIGS. 16a-16d illustrate a sequence of operation of spring loaded drives for cannula 104 and core collector 110 according to some embodiments. For clarity, handle 102 is omitted but it encloses the illustrated components except for the portions of cannula 104 and core collector 110 that protrude distally from the handle and except for the portion of drive rod 112 that extend proximally of the handle. FIG. 16a shows a spring 122 held compressed between a block 120 affixed to handle 102 and a block 112d that is affixed to drive rod 112 and can move distally and proximally along the central axis. A latch 118 holds spring 122 compressed in the position of FIG. 16a. FIG. 16b shows spring 122 released by pivoting latch 118 and expanding distally. Latch 118 can be released by operating a manual trigger, for example trigger 103 (FIG. 15). FIG. 16c shows spring 122 fully expanded, to a position in which the distal end of latch 118 has bumped the proximal end of latch 116 and has caused latch 116 to pivot to the position seen in FIG. 16c and has released spring 124 to drive block 104b and thus cannula 104 distally. A desired delay (not shown) such as a mechanical damper or buffer can be introduced between the distal end of latch 118 and the proximal end of latch 116 if it is desired to increase the time between core needle 110 reaching the end of its distal motion and the start of the distal motion of cannula 104. FIG. 16d shows the positions of the illustrated components after cannula 104 and core collector 110 have reached the end of their distal motions.

Although the foregoing has been described in some detail for purposes of clarity, it will be apparent that certain changes and modifications may be made without departing from the principles thereof. There can be many alternative ways of implementing both the processes and apparatuses described herein. Accordingly; the present embodiments are to be considered as illustrative and not restrictive, and the body of work described herein is not to be limited to the details given herein, which may be modified within the scope and equivalents of the appended claims.

1. A core biopsy instrument comprising:
a handle;
a cannula having a proximal portion secured to the handle and a distal end and configured to move distally and proximally relative to the handle along a central axis;
a drive rod having a distal portion configured to move distally and proximally along said central axis along a path coaxial with said cannula;
a core collector having a distal end, a proximal portion, and a trough intermediate said distal end and said proximal portion, wherein said trough comprises two sidewalls terminating in respective rows of teeth alternating along said central axis;
a cartridge releasably holding said core collector;
said cartridge and handle being configured for releasable attachment to each other in which said core collector extends along said central axis and is coaxial with said cannula;
said drive rod having a distal portion configured to interlock with said proximal portion of the core collector as said cartridge is being releasably attached to said handle;
said drive rod being spring loaded and configured when interlocked with said core collector to drive the core collector distally inside said cannula to a tissue sampling position in which the core collector distal end extends distally from the cannula over a selected distance;
said cannula being spring loaded and configured to respond to the drive rod having driven the core collector to said tissue sampling position to move distally over the core collector to a position at which the distal ends of the cannula and of the core collector are aligned;
said distal ends of the core collector and the cannula conforming to respective planes inclined at opposite angles to said central axis; and
said drive rod being further configured to pull said core collector proximally from said sampling position to a position in which the core collector is held in said cartridge and said cartridge being configured to be released from the handle while carrying said core collector.

2. The core biopsy instrument of claim 1, in which said teeth have proximal sides that are inclined proximally relative to said central axis.

3. The core biopsy device of claim 1, in which said core collector has holes or indentations in a proximal portion thereof and the cartridge includes one or more resilient members that hold the core collector in place in the cartridge but bend back to allow the core collector to move distally and proximally along said central axis when pushed or pulled by said drive rod interlocked therewith.

4. The core biopsy device of claim 1, in which the teeth at one side of said trough are at cross sections of the core collector at which there are no teeth on the other side of the trough so that in a cross section normal to the central axis and intersecting one of said teeth one of the sides of the trough is taller than the other.

5. The core biopsy instrument of claim 1, further including respective springs housed in said handle and configured to selectively drive distally said core collector and thereafter said cannula.

6. A core biopsy instrument comprising:
a tubular cannula extending along a central axis and having a sharp distal end;
a core collector coaxial with said cannula and configured to slide inside the cannula distally and proximally, said core collector having a distal portion, an intermediate portion and a proximal portion, wherein:
the distal portion of the core collector terminates distally in a sharp end;
the intermediate portion of the core collector comprises a trough having side walls terminating in teeth spaced apart along said central axis; and
the proximal portion of the core collector has interlock portions of differing cross sectional areas at different positions along said central axis; and
a drive rod confined to move distally and proximally along said central axis and having a distal portion with cross sections of different areas that are shaped and dimensioned to interlock with said interlock portions of the core collector.

7. The core biopsy instrument of claim 6, in which the distal end of said core collector has a distal face conforming to a plane inclined in one direction relative to the central axis and the distal end of the cannula conforms to plane included in an opposite direction to the central axis.

8. The core biopsy instrument of claim 6, further including a handle to which said core collector and said cannula are secured for proximal and distal motion along said central axis relative to the handle and to each other.

9. The core biopsy instrument of claim 6, further including a handle and a cartridge releasably secured to said handle, wherein said core collector has indentations or holes in said proximal portion thereof and said cartridge is configured to hold said core collector when aligned therewith so that the cartridge is removed from the handle together with said core collector.

10. A method comprising:
introducing a tubular cannula and a core collector into tissue while distal ends thereof are aligned along a central axis;
driving a selected length of the core collector distally from the cannula and into tissue to thereby collect an elongated sample of tissue in a trough in said core collector formed by walls terminating in spaced apart and staggered rows of teeth extending along said central axis;
thereafter, driving the cannula over said core collector to thereby sever from surrounding tissue said sample of tissue in said trough of the core collector;
pulling the core collector with said tissue sample therein proximally to a position aligned with a cartridge that is releasably secured to the handle and releasably latching the core collector to the cartridge; and
removing the cartridge with the core collector latched thereto from said handle.
